(19) United States
(12) Patent Application Publication
(10) Pub. No.: US 2006/0069320 A1
(43) Pub. Date: Mar. 30, 2006

(54) Body worn sensor and device harness

(76) Inventors: Steven B. Wolff, Pt. Roberts, WA (US); J. James Guzzetta, El Sobrante, CA (US); Gary D. Erickson, Fountain Valley, CA (US)

Correspondence Address: J. JAMES GUZZETTA, 4176 SANTA RITA ROAD, EL SOBRANTE, CA, 94803, US

(21) Appl. No.: 10/937,539

(22) Filed: Sep. 8, 2004

(51) Int. Cl. A61B 5/0402

(52) U.S. Cl. 600/509

(57) ABSTRACT

A body worn harness supporting biosensors and devices in a form fitting structure for medical long-term ambulatory operation. The supported devices provide processing, storage, and communicating of the biosensor data captured. Integrated body motion generators generate power. In addition rapid acting zero insertion force mechanical and electrical connectors are provided for the interconnection of the biosensors, devices, and power sources.

### CROSS REFERENCE TO RELATED APPLICATIONS

We claim benefit from the U.S. Provisional Application No. 60/501622 filing date Sep. 8, 2003.

### REFERENCES SITED

U.S PATENT DOCUMENTS

| | | | | | |
|---|---|---|---|---|---|
| 20040073104 | ..pickup-elect | Apr. 15, 2004 | Brun del Re | 600/372 | AdvBioelec |
| 6,721,593 | ..bodysurfac. | Apr. 13, 2004 | Anderson | 600/523 | Anderson |
| 6,605,038 | Sys..mon.. | Aug. 12, 2003 | Teller | 600/300 | Body Media |
| 6,702,755 | ..pyro/piezo.. | Apr. 9, 2004 | Stasz | 600/534 | Dymedix |
| 6,381,482 | Fabric ... | Apr. 30, 2002 | Jayaraman | 600/388 | Georgia Tech |
| 5,788,633 | ECG.. strip | Aug. 4, 1998 | Mahoney | 600/382 | HP |
| 6,400,975 | ..elec. posit.. | Jun. 4, 2002 | McFee | 600/372 | McFee |
| 6,217,525 | Redu. lead . | Apr. 17, 2001 | Medema | 600/508 | Medtronics |
| 6,494,829 | Sensor arra.. | Dec. 17, 2002 | New, Jr. | 600/300 | Nexan |
| 6,471,087 | .. garment .. | Oct. 29, 2002 | Shusterman | 600/513 | Shusterman |
| 6,751,493 | .sensor pos.. | Jun. 15, 2004 | Wenger | 600/382 | Unilead |
| 6,551,252 | . ambul. mon. | Apr. 22, 2003 | Sackner | 600/536 | VivoMetrics |
| 6,052,615 | ..four electr.. | Apr. 18, 2000 | Feild | 600/509 | Zymed |
| Not filed?? | FDA approval | ?? | ?? | ?? | Telzuit |

### STATEMENT REGARDING FEDERALLY SPONSORED RESEARCH OR DEVELOPMENT

N/A

### REFERENCE TO SEQUENCE LISTING, A TABLE, OR A COMPUTER PROGRAM LISTING APPENDIX

N/A

### FIELD OF THE INVENTION

This present invention relates to the mechanical and electrical support of external body-worn medical biosensors and their processing, storage, and communicating devices.

### BACKGROUND OF THE INVENTION

Currently cardiac analysis is performed in the hospital or clinic where the patient is resting or on a treadmill. This precludes the ability to obtain the ECG data when the patient is ambulatory. Several systems do provide improvements for the ambulatory patient. Data and event recorders such as Holter, Cardionet or King of Hearts systems provide for this. These systems typically use a belt to hold the device and use an octopus of “loose” cables to connect the device to the set of ECG electrodes.

Several cardiac diseases require longer term monitoring than is currently provided. Ischemia diagnosis ideally requires longer term monitoring in an ambulatory environment. Optimum Ischemia diagnosis requires special QRST algorithms as well.

The typical Holter system uses 3 to 5 electrodes. The typical event recorder uses 2 electrodes.

Each system has several disadvantages.

One is that the number of electrodes is often insufficient to diagnose all cardiac diseases. lschemia often requires 12 or more leads, however 12 leads are usually impractical for ambulatory operation and long term operation.

A second disadvantage is the inability to operate for long term use, that is operation beyond 48 hrs.

In addition another disadvantage is the lack of waterproof device capability.

Another disadvantage is lack of a comfortable form fitting design.

Another disadvantage is that the location of ECG electrodes requires skilled talent to locate them properly. The inaccurate placement of them results in poor data and therefore poor diagnosis.

Yet another disadvantage is the inability to support more than ECG biosensor types.

A final disadvantage is there are no real-time communications.

There are however several efforts to answer some of these disadvantages.

In order to increase the number of ECG leads Philips Zymed developed a process for a 5 electrode to 12 lead transformation, therefore allowing a minimal 5 electrode set often a simpler harness.

Medtronics also has a patent in the area of reduced lead number and transformation capability.

There are several patents covering the use of non-gel type ECG electrodes which could improve waterproof ECG biosensors. One Is Advanced Bioelectric whose patent “Enhanced pickup-electrode” covers a non-contact sensor.

Several groups have patented garment based biosensor systems, each supporting multiple biosensors, devices, and form fitting designs.

One is Vivometrics' Sackner, et al. “Systems and methods for ambulatory monitoring of physiological signs”. There design is a vest type design with an emphasis on breathing sensors.

Another is LifeShirt's Jayaramen, et al. of and Georgia Tech “Fabric or garment with integrated flexible information infrastructure for monitoring vital signs of infants”. Theirs is a fabric with emphasis on breathing and projectile sensing.

Another is New, Jr., et al. of Nexan Limited “Physiological sensor array”. Theirs has an emphasis on breathing, and also ECG.

However Cardionet, while patenting several areas in the area of real-time communications, does not have a patent in the area of biosensor body worn harness nor devices.

In addition many health-related emergency situations (i.e. heart attack, seizure, stroke, etc.) require response times of less than 1 hour to ensure chances of survival and maintenance of quality of life.

Only Cardionet provides wireless connectivity in order to obtain the benefit of a near term physician analysis of a potential L.T.E.

Therefore both the diagnostic and therapeutic efforts of the physician would benefit.

Therefore the EMR process life cycle would benefit immensely from an integrated system approach to monitoring and automatically reporting health parameter exceptions of ambulatory persons.

To date, no system or device provides all the functions required to obtain optimum health and system performance.

Prior art that is relevant to this invention are discussed below in alphabetical order of their companies (assignees).

Brun del Re, Riccardo; et al. of Advanced Bioelectric in 20040073104 “Enhanced pickup-electrode” teaches about an active electronic non-gel type ECG electrode.

Anderson in 6,721,593 “Apparatus for body surface mapping” illustrates the Body Surface Mapping system, which typically has a large number of electrodes (from 32 to 512).

Teller, et al. in 6,605,038 of BodyMedia “System for monitoring health, wellness and fitness” illustrates other body worn sensor systems. In this patent they show a wrist mounted sensor and device system.

Stasz, et al. of Dymedix, Corp. in 6,702,755 “Signal processing circuit for pyro/piezo transducer” teaches about breathing sensors utilizing pyro and/or piezoelectric materials.

Jayaramen, et al. of Life Shirt and Georgia Tech in 6,381,482 “Fabric or garment with integrated flexible information infrastructure for monitoring vital signs of infants” teaches about weaving a fabric of sensors into a vest like garment for vital signs monitoring and “penetration” detection.

Mahoney of HP 5,788,633 in “ECG electrode strip with elongated slots” teaches about adjustable ECG sensor location on the patients body. This is one of the first attempts at accommodating a variety of patient sizes.

McFee in 6,400,975 “Apparatus and method for consistent patient-specific electrode positioning for EKG testing and delivery of electrical energy to the heart” illustrates a mechanism for locating and locking an array of ECG electrodes and the reproducibility therein.

Medema, et al. of Medtronic Physio-Control Manufacturing Corp. (Redmond, Wash.) in 6,217,525 “Reduced lead set device and method for detecting acute cardiac ischemic conditions” is one approach to diagnosing cardiac Ischemia using a reduced lead set and their version of cardiac signal classification system.

New, Jr., et al. of Nexan Limited (Cambridge, GB) in 6,494,829 “Physiological sensor array” illustrates a sensor system including respiration, and also ECG.

Shusterman in 6,471,087 “Remote patient monitoring system with garment and automated medication dispenser” teaches about a garment based an integrated remote patient monitoring system that includes the garment, a monitoring device, and a medication dispensing unit.

Wenger of Unilead International, Inc. (Lafayette, Calif.) in 6,751,493 “Universal electrocardiogram sensor positioning mask with reposition-able sensors and method for employing same” teaches about a template methodology for locating ECG electrodes on many sizes of patients with a single harness template. The patent mainly applies to the precordial leads (V1 to V6) of the 12 lead set.

Sackner, et al. of VivoMetrics, Inc. (Ventura, Calif.) in 6,551,252 “Systems and methods for ambulatory monitoring of physiological signs” illustrates another monitoring garment comprising a shirt for the torso of the patient or individual. In addition they teach about an inductive plethysmographic (IP) sensors that one of the sensors supported in the vest.

Feild, et al. of Zymed Medical Instrumentation, Inc. (Camarillo, Calif.) in 6,052,615 “Method and apparatus for sensing and analyzing electrical activity of the human heart using a four electrode arrangement” teaches a method for transforming standard electrodes at the E, A, S, I locations into a 12 lead waveform set.

Telzuit of Florida in a FDA approval document discusses a ECG harness for EASI and 12 lead sub-set locations with templates in 4 sizes each, implying a total of 8 harnesses.

None of this prior art teaches the following:

The support of a plurality of biosensors on a form fitting harness.

The connecting of the devices to the harness with a water proof rapid attaching (ZIF) connector.

The support of, but not limited to, ECG, EEG, blood pressure, oxygen, breathing, sound, and motion sensors.

The support of standard ECG electrode, and their water resistant connectors, as well as the use of dry contact ECG sensors, such as capacitive, active, or optical types.

The recording and/or processing of biosensor signals.

Utilizing User Interface elements on the device, which are visible to the patient.

Affixing a device over a standard ECG electrode patch in order to increase the harness's weight resistance capacity.

Positioning a device over the body's skin surface for direct sensor access.

The transformation of the ECG biosensors' signals within the device into that of a variety of alternative ECG lead set waveforms.

Utilizing in the harness water resistant, reusable or disposable, sterilize-able design and materials.

The use of an adhesive backing along the body side of the form-fitting harness, in discrete positions or continuously.

Producing electrical power from a body motion powered generator integrated into the harness, where the generator utilizes power generation materials, but not limited to, piezoelectric, pyroelectric, magnetostrictive materials et al.

Sealing standard ECG connectors from any water, food, and body fluids

Utilizing a single harness for all adult body sizes for the locations of the ECG biosensors.

Utilizing a different harness for each of the lead sets such as optimum lead, EASI lead, as well as the Precordials for the 12 leads positions.

### SUMMARY OF THE INVENTION

Accordingly, the present invention provides the functionality of a form fitting medical biosensor and device harness that supports long term monitoring.

The present invention provides for a plurality and variety of biosensors to be accurately located on the patient's body. The biosensors can include ECG, EEG, blood pressure, oxygen, breathing, sound, and motion types sensors.

In accordance with a further aspect of the present invention, the devices provide processing, storage, and communication. In addition some biosensors can be used for power generation.

The novel elements believed to be characteristic of the present invention are set forth in the claims.

### BRIEF DESCRIPTION OF THE SEVERAL VIEWS OF THE DRAWINGS

### Drawings

FIG. 1 Harness

FIG. 2 Device

FIG. 3 Sensors

FIG. 4 Power

FIG. 1 Harness

The support of a plurality of biosensors and their processing devices on a form fitting harness.

FIG. 2 Device

Devices are connected to biosensors' signals and provide signal processing and User Interface

FIG. 3 Sensors

A variety biosensors' are supported

FIG. 4 Power

Power is generated from a motion biosensor

### REFERENCE NUMERALS IN DRAWINGS

FIG. 1 Harness
biosensors (1)
form fitting (2)
harness (3)
water resistant (4),
reusable or disposable, sterilize-able design and materials (5)
adhesive backing (6)
adhesive in discrete positions (7)
continuous adhesive (8).
sealed connectors (9)
standard ECG connectors (10)
single harness for all adult body sizes (11)
each of the lead sets (12)
optimum lead (13)
EASI lead (14)
Precordials for the 12-lead positions (15)

FIG. 2 Device
device connections (16) to the harness
water proof rapid attachment connector (17)
device affixed over a standard ECG electrode patch (18)
device located over the body's surface for direct sensor access (19)
recording (20)
processing (21)
biosensor signals (22)
transformation (23) of the ECG biosensors' signals
alternative ECG lead set (24) waveforms
User Interface elements (25)
User Interface visible by the patient (26).

FIG. 3 Sensors
ECG (27)
EEG (28)
breathing (29)
pulse oxyimetry (29a)
blood pressure (29b)
sound (30)
motion sensors (31)
standard ECG electrode (32)
water resistant connectors (33)
dry contact ECG sensors (34)
capacitive, active, or optical types ECG sensors (35)

FIG. 4 Power
electrical power production (36)
body motion powered generator (37)
generator integrated into the harness (38)
piezoelectric, pyroelectric, magnetostrictive materials (39)

### DETAILED DESCRIPTION OF THE INVENTION

The present invention provides the functionality of a form fitting medical biosensor and device harness that will support long term monitoring.

The present invention provides for a plurality and variety of biosensors to be accurately located on the patient's body. The biosensors can include ECG, EEG, breathing, blood pressure, oxygen, sound, and motion types sensors.

In accordance with a further aspect of the present invention, the devices provide processing, storage, and communication. In addition some biosensors can be used for power generation.

Harness

The present invention provide for the support of a plurality of biosensors (1) on a form fitting (2) body worn harness (3).

The harness is designed for and constructed from water resistant (4), and sterilize-able materials (5). In addition the harness may be provided as reusable and/or in disposable versions.

An adhesive backing is used (6) along the body side of the form-fitting harness, either in discrete positions (7) or continuously (8).

In one embodiment standard ECG connectors (10) may be sealed (9) from any water, food, and body fluids.

A single harness is provided for all adult body sizes for the locations (11) of the ECG biosensors by using a set of several ECG locations on a single harness that represent the diversity of adult body sizes.

In another embodiment a different harness is provided for each of the various lead sets (12) such as optimum lead (13), EASI lead (14), as well as the Precordials (15) for the 12-lead positions.

Device

The device or devices (16) are connected to the harness by a waterproof and rapid attachment connector (17).

In one embodiment a device is positioned over a standard ECG electrode patch (18) thereby increasing the harness's weight resistance capacity.

In another embodiment a device is positioned over the body's surface for direct sensor access (19) such as for a pulse oximetry sensor.

The device or devices provide for recording (20) and/or processing (21) of biosensor signals (22).

In another embodiment a lead transformation process (23) within the device transforms the ECG biosensors' signals from the native lead number and location map into that of a variety of alternative ECG lead set (24) waveforms.

Another embodiment utilizes User Interface elements (25) on the device that are visible by the patient (26).

Sensors

In several embodiments various sensors are supported. These may include ECG (27), EEG (28), breathing (29), pulse oxyimetry (29a), blood pressure (29b), sound (30), and motion (31) sensors.

In one embodiment standard ECG electrodes (32) use water resistant connectors (33).

In another embodiment dry contact ECG sensors (34) are utilized such as capacitive, active, or optical types (35).

In an additional embodiment the processing of vibration information for several sensor displacement modes is accomplished simultaneously from the same biosensor. The described integrated sensor utilizes multiple materials within the same sensor to detect optical, magnetostrictive, piezoelectric, pyroelectric and capacitive modalities.

Power

In a embodiment electrical power (36) is produced from a body motion powered generator (37) which is integrated into the harness (38), and where the generator utilizes power generation materials, such as piezoelectric, pyroelectric, and magnetostrictive materials (39) for example.

What is claimed is:

1. A method of electro-mechanically supporting a plurality of biosensors and computing device or devices communicating and processing said biosensors' signals;
mounted on the body of an ambulatory patient comprising;
supporting a plurality of biosensors on a form fitting harness;
locating the biosensors accurately with a biosensor location template;
connecting the biosensors to the device or devices with embedded cables;
connecting the device or devices with a waterproof rapid attaching connector.

2. The method of claim 1, wherein the step of supporting a plurality of biosensors comprises:
supporting of, but limited to, ECG, EEG, oxygen, breathing, sound, and motion sensors.

3. The method of claim 1, wherein the step of supporting ECG biosensors comprises:
interfacing to standard ECG electrode and their water resistant connectors;
interfacing to dry contact ECG sensors such as capacitive, active, and optical types.

4. The method of claim 1, wherein the step of devices communicating and processing said biosensors' signals comprises:
communicating via wireless and/or wired devices;
recording and/or processing said biosensor signals;
displaying user interface elements usable by the patient;
affixing said device to the harness and located over a standard ECG electrode patch increasing said device weight resistant capacity;
positioning said device over the body's surface for direct sensor access;
transforming said ECG biosensors' signals within the device into that of a variety of alternative ECG lead set waveforms.
Dependy

5. The method of claim 1, wherein the step of mechanically supporting a plurality of biosensors and devices comprises:
contouring on the body a form-fitted, secure, and comfortable harness;
utilizing_water resistant, reusable or disposable, sterilize-able harness design and materials;
applying an adhesive backing in discrete positions or continuously along the body side;
utilizing the adhesive power of standard ECG electrodes patch to support the harness.

6. The method of claim 2, wherein the step of supporting a plurality of biosensors comprises:
receiving electrical power from a body motion powered generator;
integration of said body motion powered generator within said harness;
utilizing power generation materials, including, but not limited to, optical, piezoelectric, pyroelectric, capacitive and magnetostrictive;
sensing materials can be utilized separately or in combination.

7. The method of claim 3, wherein the step of supporting ECG biosensors comprises:
sealing said standard ECG electrode connector from any water, food, and body fluids.

8. The method of claim 1, wherein the step of sensor location template comprises:
locating said ECG biosensors' locations on the body for optimum lead, EASI lead, and the Precordial leads;
utilizing a single harness for all adult body sizes;
deploying each lead set in different harnesses.

9. The method of claim 4, wherein the steps of alternative ECG lead sets comprising:
selecting one of, but not limited to, optimum lead, EASI lead, and Precordials for 12 leads.

10. The method of claim 1, wherein the step of supporting ECG biosensors comprises:
connecting said biosensors to said device(s) with embedded cables.

11. The method of claim 1, wherein the step of supporting the devices comprises:
connecting said device or devices with a waterproof rapid attaching connector.
